(19) United States
(12) Patent Application Publication
(10) Pub. No.: US 2017/0058277 A1
(43) Pub. Date: Mar. 2, 2017

(54) COMPOSITE LIQUID CELL (CLC) SUPPORTS, AND METHODS OF MAKING AND USING THE SAME

(71) Applicant: GenCell Biosystems Ltd., Raheen (IE)

(72) Inventor: Mark Dalton, Limerick (IE)

(21) Appl. No.: 15/211,471

(22) Filed: Jul. 15, 2016

Related U.S. Application Data
(60) Provisional application No. 62/210,298, filed on Aug. 26, 2015.

(51) Int. Cl. C12N 15/10; G01N 35/10; G01N 35/00; C12Q 1/68; B01L 3/00

(52) U.S. Cl. CPC C12N 15/1075; C12Q 1/6874; B01L 3/5085; B01L 3/50851; G01N 35/0099; G01N 35/1002; B01L 2300/0829; B01L 2300/0851; B01L 2300/069; B01L 2200/12

(57) ABSTRACT

Composite liquid cell supports are provided. Aspects of the supports include: a plurality of CLC containers, wherein each CLC container is configured to hold a CLC and comprises a fluorophilic inner surface having a water contact angle of 80 degrees or greater. The fluorophilic inner surface may have a first contact angle with a fluorous carrier liquid which is less than a second contact angle with an encapsulating liquid that is immiscible with the carrier liquid. The supports find use in, among other applications, CLC systems and devices. Also provided are methods of preparing and using CLC arrays that include the CLC supports of the invention.

### CROSS-REFERENCE TO RELATED APPLICATIONS

Pursuant to 35 U.S.C. §119(e), this application claims priority to the filing date of U.S. Provisional Patent Application No. 62/210,298, filed Aug. 26, 2015; the disclosure of which application is herein incorporated by reference.

### INTRODUCTION

Processing of biological samples can be advantageously done within a fluid system involving three mutually immiscible liquids. Such a system can be used to create composite liquid cells (CLCs) in which a sample fluid is isolated by an encapsulating fluid, and both of which float on top of a carrier fluid. CLCs are described in more detail in U.S. Pat. No. 8,465,707, which is hereby incorporated herein by reference in its entirety.

In some implementations, CLCs are centered around an aqueous phase (also referred to as a micro-reactor) which contains a sample or reagent of interest, e.g., a biological component or reagent. The aqueous phase floats on top of a carrier fluid that is immiscible with, and more dense than, the aqueous phase. Above the aqueous phase is an encapsulating fluid that is immiscible with both the aqueous phase and the carrier fluid, and is less dense than both water and the carrier fluid. In some instances, the aqueous phase is completely surround by the encapsulating fluid, such that it is does not directly contact the carrier fluid. In this way a CLC is “triphasic”, that is, it includes three mutually immiscible phases: a carrier fluid, an aqueous phase (sometimes called a sample) and an encapsulant. CLCs have proven to be robust and can be manipulated, e.g., moved from one location to another, added to, merged with other CLCs, split, etc. Encapsulation leaves CLCs essentially free of contamination. CLCs can also be formed down to very small sizes, and the small volumes involved allow for highly efficient use of potentially expensive reagents.

All these factors mean that CLCs are excellent venues for biological sample processing, for example, in PCR, dPCR, qPCR, TMA, bDNA, LCR, and nucleic acid library preparation.

While CLCs can be formed on the free surface of a large carrier liquid bath, triphasic arrangements of fluids can also be generated, stored, or otherwise located inside a small, self-contained vessel (or well).

### SUMMARY

Composite liquid cell supports are provided. Aspects of the supports include: a plurality of CLC containers, wherein each CLC container is configured to hold a CLC and comprises a fluorophilic inner surface having a water contact angle of 80 degrees or greater. The fluorophilic inner surface may have a first contact angle with a fluorous carrier liquid which is less than a second contact angle with an encapsulating liquid that is immiscible with the carrier liquid. The supports find use in, among other applications, CLC systems and devices. Also provided are methods of preparing and using CLC arrays that include the CLC supports of the invention.

### BRIEF DESCRIPTION OF THE FIGURES

The invention may be best understood from the following detailed description when read in conjunction with the accompanying drawings. Included in the drawings are the following figures:

FIG. 1 provides a schematic of a CLC contained in an exemplary container (100) having an inner surface (101) in contact with a carrier liquid (104). Disposed on a top surface of the carrier liquid is an encapsulating liquid (102) whose interface with the carrier liquid defines a meniscus (105) and inside which is contained an aqueous sample liquid or micro-reactor (103).

FIG. 2 shows an image of an exemplary multiwell CLC system. The multiwell support (200) includes an encapsulating liquid (202) disposed on the top surface of a carrier liquid (204) whose interface defines a visible concave meniscus (205).

FIG. 3 shows an image of the exemplary multiwell CLC system of FIG. 2 (300) with an aqueous sample liquid in place (303) in the encapsulating liquid of the CLC in three wells. The aqueous sample liquid includes a dark colored dye component to aid in visualization. The image shows that the aqueous sample liquid is self-centered in the container away from the inner surface (401) of the CLC container.

FIG. 4 shows a schematic detailing the dimensions of the exemplary multiwell support of FIGS. 2 and 3 that was prepared using an injection moulding procedure with a fluoropolymer. Unless otherwise specified, dimensions and tolerances are in millimeters. Tolerances are as follows: ±0.1 mm, ≦±0.5°.

FIG. 5A shows schematic images detailing the dimensions of another exemplary multiwell support. Unless otherwise specified, dimensions and tolerances are in millimeters. FIG. 5B shows an image of the exemplary multiwell CLC system of FIG. 5A with an aqueous sample liquid in place in the encapsulating liquid of the CLC in three wells. The aqueous sample liquid includes a dark colored dye component to aid in visualization. The image shows that the aqueous sample liquid is self-centered in the container away from the inner surface of the CLC container.

FIG. 6 shows the results of contact angle measurements of a material of interest that finds use in exemplary CLC supports. FEP, a common fluoropolymer was used as a reference material as it was known to provide the appropriate surface properties for CLCs. The results from this study were used to define the contact angles between a fluorous carrier fluid (GC1) and the housing material and between the encapsulating silicone oil (GC2) and the housing material. The moulded (injection moulded polypropylene) data set was known to yield the incorrect meniscus shape. Analysis of the two data sets enabled inference of the critical properties for CLCs.

### DETAILED DESCRIPTION

Composite liquid cell supports are provided. Aspects of the supports include: a plurality of CLC containers, wherein each CLC container is configured to hold a CLC and comprises a fluorophilic inner surface having a water contact angle of 80 degrees or greater. The fluorophilic inner surface may have a first contact angle with a fluorous carrier liquid which is less than a second contact angle with an encapsulating liquid that is immiscible with the carrier liquid. The supports find use in, among other applications, CLC systems and devices. Also provided are methods of preparing and using CLC arrays that include the CLC supports of the invention.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, the CLC supports are first described in more detail. Next, systems and devices in which the subject supports find use are described. Then methods of preparation and use of the CLC systems are described.

### CLC Supports

As summarized above, aspects of the invention include composite liquid cell (CLC) supports. A CLC support includes at least one CLC container that is configured to hold a CLC. The term CLC is used to refer to a triphasic fluid arrangement which is a combination of at least three substantially mutually immiscible fluids having three different densities. The first fluid is a carrier fluid which is the densest of the three substantially mutually immiscible fluids; the second fluid is an encapsulating fluid which is the least dense of the substantially mutually immiscible fluids; and the third fluid is a target fluid (sometimes referred to as a “sample”) which has a density that is less than the first fluid and greater than the second fluid. Thus, in a triphasic fluid arrangement, a core aqueous liquid (which may be made up of a sample and may be referred to as a micro-reactor, such as described in greater detail below, is encased (or encapsulated) between the carrier fluid and the encapsulating fluid. In certain embodiments, the aqueous fluid contains a biological sample, reagent, buffer, or other prescribed element of a biological assay or biochemical protocol. Examples of components that can be present in the aqueous fluid include, but are not limited to: cells, nucleic acids, proteins, enzymes, biological sample (e.g., blood, saliva, etc.), buffers, salts, organic material, and any combination thereof. Depending on the particular CLC, the aqueous liquid may or may not directly contact the carrier liquid, and may in some instances be present in a carrier liquid that assumes a roughly spherical shell about the aqueous liquid, which shell rests or floats on a surface of the carrier liquid. Additional details regarding carrier, encapsulating and target fluids may be found in U.S. Pat. Nos. 8,465,707 and 9,080,208; as well as United States Patent Application Publication No. 20140371107; and Published PCT Application Nos: WO2014/083435; WO2014/188281; WO2014/207577; WO2015/075563; WO2015/075560; the disclosures of which applications are herein incorporated by reference.

The present disclosure provides a CLC container having an inner surface configured to make contact with and to contain a CLC. Unless otherwise specified, the term “CLC” as used herein refers to both the encapsulating liquid droplet with microreactor and the carrier liquid upon which the encapsulating liquid is disposed. The inner surface properties of the CLC container provide a desirable concave meniscus shape with the carrier liquid which ensures containment of the micro-reactor of the CLC in a desirable and consistent location at the center of the container. In some cases, the micro-reactor of the CLC self-positions at the center bottom of an encapsulating liquid droplet which is disposed on the top concave surface of the carrier liquid. Such consistent positioning in a particular relative location is advantageous for performing automated processes involving addition and removal of aqueous liquid to and from the micro-reactor.

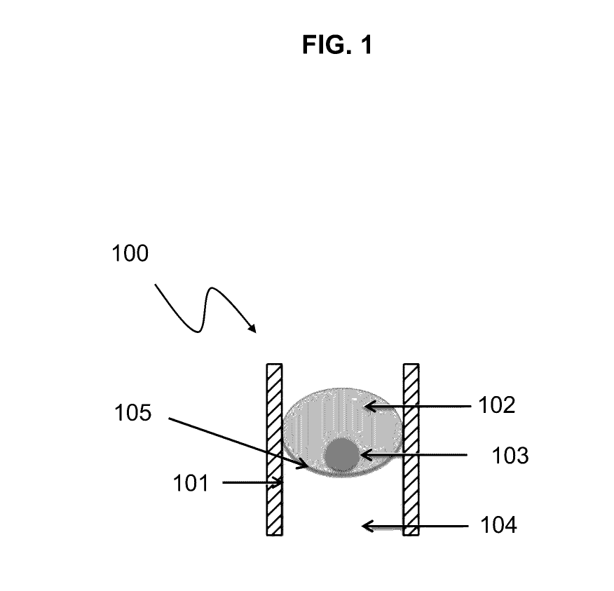

FIG. 1 illustrates a schematic of an exemplary CLC container holding a CLC. The depicted CLC container (100) has side walls having an inner surface (101) in contact with the carrier liquid (104) of the CLC. Disposed on the top surface of the carrier liquid is an encapsulating liquid (102) inside which an aqueous sample liquid (103) is contained. The interface of the carrier liquid and the encapsulating liquid defines a meniscus (105) that extends between the walls of the container. The concave shape of the meniscus is determined by the wettability of the inner surface of the container with the immiscible carrier and encapsulating liquids. The inner surface may have a high affinity for (e.g., high wettability with) the carrier liquid that provides a concave meniscus. In some instances, the inner surface is fluorophilic (e.g., as described herein) and the carrier liquid is a fluorous carrier liquid. In some cases, the inner surface of the CLC container has higher wettability for the carrier liquid over the encapsulating liquid.

As used herein, the term “contact angle” refers to the angle, measured through the liquid, where a liquid/vapor interface meets a solid surface. The contact angle is used to quantify the wettability of a solid surface by a liquid via the Young equation. For example, when water is the liquid, the term “hydrophobic” may be applied to surfaces which give a contact angle of 90 degrees or greater. The term “hydrophilic” may be applied to surfaces which give a contact angle of less than 90 degrees. It is understood that the degree of hydrophobicity or hydrophilicity of a surface may vary with the water contact angle. The term “superhydrophobic” is applied to surfaces which give contact angles at least 150 degrees with water. For a perfectly hydrophobic surface the contact angle should be 180 degrees.

The subject CLC containers have an inner surface which is sufficiently hydrophobic to ensure that no wetting occurs of the container walls by the aqueous sample liquid of the CLC. Preventing surface wetting by the aqueous sample liquid is desirable to ensuring high recovery of micro-volumes of samples of interest from the CLC micro-reactors. In some embodiments, the inner surface of the CLC container has a water contact angle of 80 degrees or more, such as 85 degrees or more, 90 degrees or more, 95 degrees or more, 100 degrees or more, 105 degrees or more, 110 degrees or more, 115 degrees or more, 120 degrees or more, or even more. In certain embodiments, the inner surface has a water contact angle of 80 degrees or more. In some cases, the inner surface has a water contact angle ranging from 80 to 150 degrees, such as from 80 to 120 degrees, 80 to 110 degrees, 80 to 100 degrees, or 80 to 90 degrees. In certain cases, the inner surface is hydrophobic and has a water contact angle ranging from 90 to 150 degrees, such as from 90 to 130 degrees, 90 to 120 degrees, 90 to 110 degrees or 90 to 100 degrees.

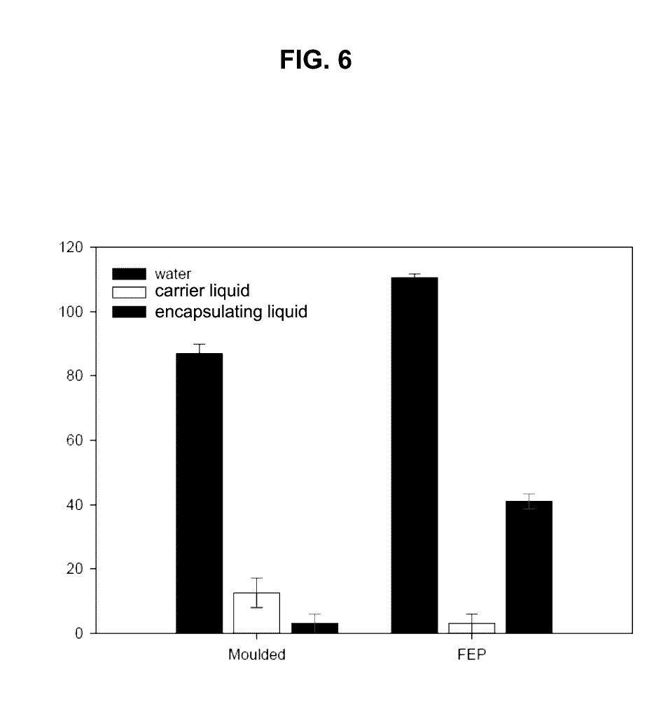

In addition, the inner surface of the CLC container has relatively high wettability for the carrier liquid versus the encapsulating liquid. The relative wettability may be compared via contact angles. A relatively high wettability of the inner surface with a liquid may be defined by a low contact angle. In some cases, the inner surface has a first contact angle with the carrier liquid of a CLC that is less than a second contact angle with the encapsulating liquid of a CLC. In some embodiments, the inner surface of the CLC container is fluorophilic (e.g., as described herein) and provides for preferential wetting of the surface by a fluorous carrier liquid relative to a non-fluorous encapsulating liquid. In some cases, the inner surface has a level of fluorophilicity that provides for a low contact angle (i.e., high wettability) with a fluorous carrier liquid relative to the encapsulating liquid. For example, FIG. 6 shows the contact angles of an exemplary fluoropolymer surface (FEP) for water, carrier liquid and encapsulating liquid. (Carrier oil=GC1, Encapsulating oil=GC2, Moulded=injection moulded polypropylene)

In some embodiments, the inner surface of the CLC container has a contact angle of 20 degrees or less for the carrier liquid, such as 15 degrees or less, 10 degrees or less, 9 degrees or less, 8 degrees or less, 7 degrees or less, 6 degrees or less, 5 degrees or less, 4 degrees or less, 3 degrees or less, 2 degrees or less or even 1 degree or less. In certain embodiments, the inner surface has a contact angle with the carrier liquid of 5 degrees or less. In some instances, the contact angle ranges from 1 to 20, such as 2 to 15, e.g., 3 to 10.

The inner surface of the CLC container may have a contact angle with the encapsulating liquid that is greater than the contact angle with the carrier liquid, as described herein. In some embodiments, the inner surface of the CLC container has a contact angle of 20 degrees or more with the encapsulating liquid, such as 25 degrees or more, 30 degrees or more, 35 degrees or more, 40 degrees or more, 45 degrees or more, 50 degrees or more, 55 degrees or more, 60 degrees or more, 65 degrees or more, 70 degrees or more, etc. In certain embodiments, the inner surface has a contact angle with the encapsulating liquid that ranges from 20 to 80 degrees, such as 25 to 75 degrees, e.g., 35 degrees. In some embodiments, the inner surface of the CLC container has a contact angle with the encapsulating liquid that is at 10 degrees or greater than the contact angle with the carrier liquid, such as 15 degrees or greater, 20 degrees or greater, 25 degrees or greater, 30 degrees or greater, 35 degrees or greater, 40 degrees or greater, 45 degrees or greater, 50 degrees or greater, 55 degrees or greater, 60 degrees or greater, 65 degrees or greater, 70 degrees or greater, etc., where in some instances the magnitude of the difference ranges from 10 to 90 degrees, such as 15 to 75 degrees.

In certain embodiments of the inner surface of the CLC container, the water contact angle is greater than the encapsulating liquid-contact angle, which are both greater than the carrier liquid-contact angle. In some instances, the inner surface has a contact angle with the encapsulating liquid that is less than the water contact angle, such as 20 degrees or less, 25 degrees or less, 30 degrees or less, 35 degrees or less, 40 degrees or less, 50 degrees or less, 60 degrees or less, 70 degrees or less, 80 degrees or less than the water contact angle, where in some instances the magnitude of the difference ranges from 10 to 90 degrees, such as 15 to 75 degrees.

As used herein, the terms “fluorous” and “fluorinated” are used interchangeably and refer to a substance (e.g., a liquid, a surface, a material, a substrate, etc.) that includes a compound having at least one fluorine-substituted carbon atom. In some embodiments, a fluorinated compound includes a branched or unbranched, fluorinated C₁-C₁₈ alkyl group, a branched or unbranched, fluorinated C₂-C₁₈ alkenyl group, a fluorinated cycloalkyl group, a fluorinated cycloalkylalkylene group, a branched or unbranched, fluorinated C₂-C₁₈ alkynyl group, a fluorinated aryl group, or a fluorinated arylalkylene group. In certain instances, the fluorinated compound includes a perfluorinated group, such as an alkyl group that is perfluorinated. By “perfluorinated” is meant that all available hydrogens of a group of interest have been substituted with fluorine atoms.

### Fluorophilic Inner Surface

The present disclosure provides supports and containers that have an inner surface configured to make contact with a CLC disposed therein. The materials of the inner surface may be selected to provide for desirable wetting properties with a carrier liquid of interest. In some embodiments, the inner surface of the CLC container is a fluorophilic surface. Fluorophilicity refers to the affinity of one substance for another fluorinated substance and is in some cases based on the affinity that fluorinated compounds, e.g., fluorocarbons and fluorohydrocarbons, have for each other. As such, in some cases, a fluorophilic substance is itself fluorinated. A fluorophilic substance may be hydrophobic or hydrophilic depending on its composition. In some cases, fluorophilicity may be expressed in terms of the partition coefficient (InP) of a molecule between equal volumes of perfluoromethylcyclohexane and toluene. In some cases, a fluorophilic surface is one which has high wettability for a fluorous solvent (e.g., a contact angle of 90 degrees or less, such as 45 degrees or less, 20 degrees or less, or even less with a fluorous solvent of interest (e.g., perfluoromethylcyclohexane).

A “fluorophilic surface” is meant to include a material having at least one component that is fluorinated (e.g., as described herein) and provides for fluorophilicity, e.g., affinity for a fluorous carrier liquid. Any convenient fluorinated materials may be utilized in the subject supports to provide for a fluorophilic surface. A variety of fluorous materials and liquids are available in the fields of solid phase fluorous extraction and fluorous liquid-liquid extraction which may be adapted for use in the subject systems. A variety of fluorinated coatings and polymers may be adapted for use in the inner surfaces of the subject substrates.

In some cases, the support comprises a plurality of containers having fluorophilic inner surfaces that are composed of a different material than the underlying support material (e.g., the support materials may be heterogeneous). In other instances, the underlying support and the fluorophilic inner surface of the plurality of containers are composed of the same material. In such cases, the support may be referred to as composed of a homogeneous material, which may provide for a desirable and simplified manufacturing process. In some instances, a polymeric support has a fluorination modified surface, e.g., a polymer surface that has been treated with fluorine gas to fluorinate the outer layer. In some instances, the plurality of containers is structurally connected to each other by the underlying support, e.g., the support has an integrated structure, such as is found in a multiwell plate.

In certain embodiments, the fluorophilic inner surface is composed of a fluoropolymer. Any convenient fluoropolymers may be utilized in the subject supports to provide for a fluorophilic inner surface. In certain instances, the fluoropolymer is a melt processible fluoropolymer or cofluoropolymer. Melt processible fluoropolymers or fluoroplastics find use in a variety of fabrication techniques such as injection molding, wire, tube, and film extrusion, rotational molding, blow molding, compression molding, and transfer molding, any of which may be adapted for use in the fabrication of the subject supports. Fluoropolymers of interest include, but are not limited to, tetrafluoroethylene homopolymers or copolymers, chlorotrifluoroethylene (CTFE) homopolymers or copolymers and vinylidene fluoride (VDF) homopolymers or copolymers. In certain instances, the fluorophilic inner surface comprises polytetrafluoroethylene. In certain cases, the fluorophilic inner surface comprises a co-polymer of tetrafluoroethylene and hexafluoropropylene. In some instances, the fluorophilic inner surface comprises a copolymer of tetrafluoroethylene and perfluoroalkyl vinylether. In some embodiments, the fluorophilic inner surface comprises a copolymer of tetrafluoroethylene and ethylene. Any convenient additives may be included in the subject fluoropolymers, including but not limited to, surfactants, pigments, antioxidants, stabilizers, fillers, vulcanization coagents, etc. Such additives may provide for a desirable property such as an optical property or physical property that is desirable for fabrication. In some embodiments, the fluoropolymer further comprises a pigment that makes the support opaque. In certain instances, the fluoropolymer further comprises a black pigment or black colorant that provides for desirable low backgrounds in applications involving luminescence.

Exemplary fluoropolymers of interest include, but are not limited to, POLYFLON™ PTFE (e.g., M-series, F-series or D-series) or polytetrafluoroethylene, NEOFLON™ FEP or melt processable perfluoro co-polymer of tetrafluoroethylene and hexafluoropropylene, NEOFLON™ PFA or copolymer of tetrafluoroethylene and perfluoroalkyl vinylether, NEOFLON™ ETFE or copolymer of tetrafluoroethylene and ethylene, and the like. In certain instances, the fluoropolymer is a copolymer of tetrafluoroethylene and perfluoroalkyl vinylether. In certain embodiments, the subject support has an integrated structure defining multiple CLC containers, which is prepared via an injection molding procedure using a fluoropolymer (e.g., as described herein).

The supports of the present disclosure can be manufactured according to any convenient fabrication techniques including, but not limited to, as injection molding, wire, tube, and film extrusion, rotational molding, blow molding, compression molding, solvent casting and transfer molding, any of which may be adapted for use in the fabrication of the subject supports. In some instances, the support is an integrated support composed of a homogeneous material such as a melt processible fluoropolymer or fluoroplastic which has been injection moulded to provide a convenient multi-well configuration.

### Support Configurations

The subject supports may have any desirable configuration of CLC container(s). In some embodiments, the support includes a single container. In some instances, the support includes a plurality of discrete CLC containers that are fluidically independent from one another (e.g., discrete containers do not share carrier liquid). The subject supports including discrete containers provide for individual processing of the CLCs in the containers. For example, each CLC may be independently thermally controlled, e.g., for applications where thermal cycling of a CLC microreactor is desirable.

In some instances, the support includes, 2 or more, such as 3 or more, 4 or more, 5 or more, 6 or more, 8 or more, 10 or more, 12 or more, 14 or more, 16 or more, 17 or more, 24 or more, 48 or more, 96 or more, or even more discrete CLC containers. In some cases, the supports may include standard-sized CLC containers and can include multiple, discrete, individual CLC containers arranged in a two-dimensional grid, e.g., a grid of 8, 12, 16, 24, 48, 96, 384, 1536 or 3456 containers, in rows, such as 2 or more rows, e.g., 3 or more rows, or any other convenient configuration. The present disclosure provides for support materials that may be easily formed into any convenient support shape and CLC container configurations. As used herein, the term “support” is meant to include both the CLC container(s) themselves and the underlying solid structural scaffold in which the container(s) are configured or housed, e.g., in a particular multi-well array.

A CLC container may have any convenient shape. The CLC container may have a cylindrical shape of any convenient diameter and of any convenient height. In some cases, the CLC container is a tube. A tube container can have any convenient shaped bottom, such as a flat, rounded or conical bottom. In certain instances, the CLC container is a well, such as a chimney well or a rounded well. In some cases, the support is configured in a multi-well format, such as a multi-well plate. The multi-well plate format may allow for use of the CLC support in a variety applications including common assay formats for pharmaceutical high-throughput screening laboratories, molecular biology research laboratories, and diagnostic assay laboratories where microtiter plates, automated liquid handling and optical plate readers find use. The subject containers may have any convenient volume. In some cases, the container has a volume ranging from 10 μL to 10 mL, such as 30 μL to 500 uL. In certain instances, the CLC container has a volume of 10 mL or less, such as 5 mL or less, 2 mL or less, 1.5 mL or less, 1.0 mL or less, 0.7 mL or less, 0.5 mL or less, 0.2 mL or less.

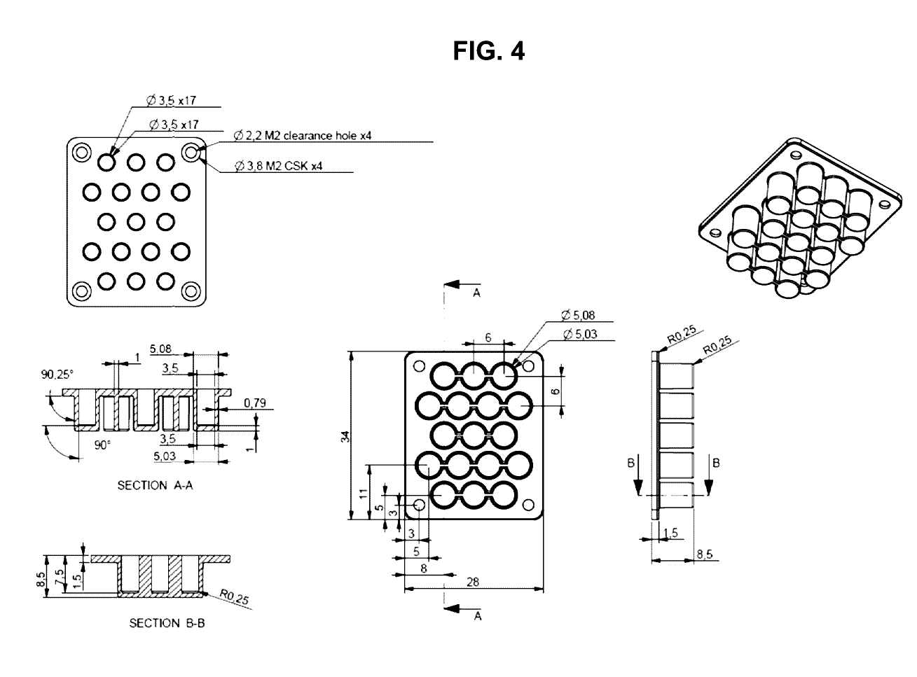

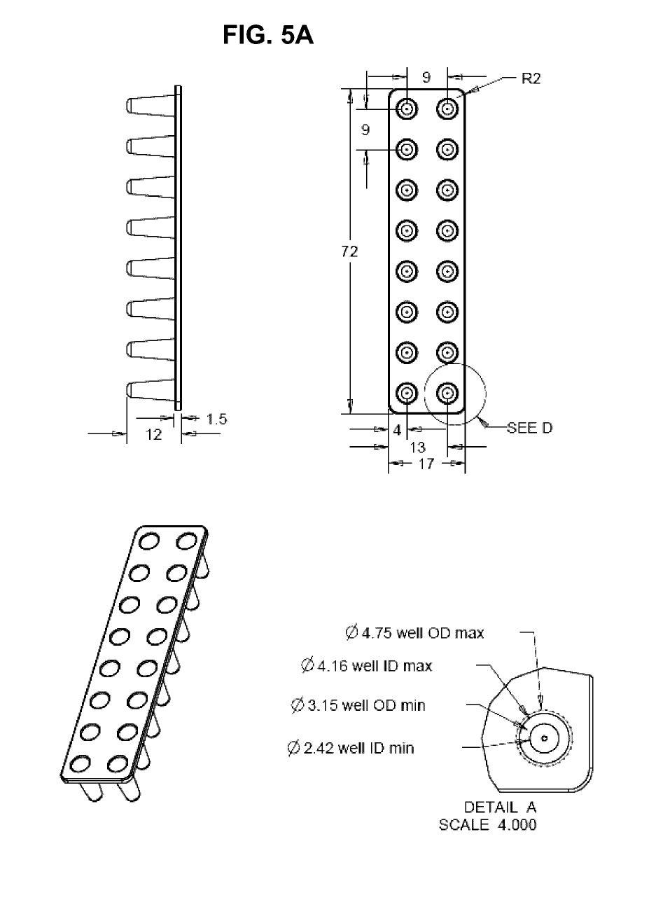

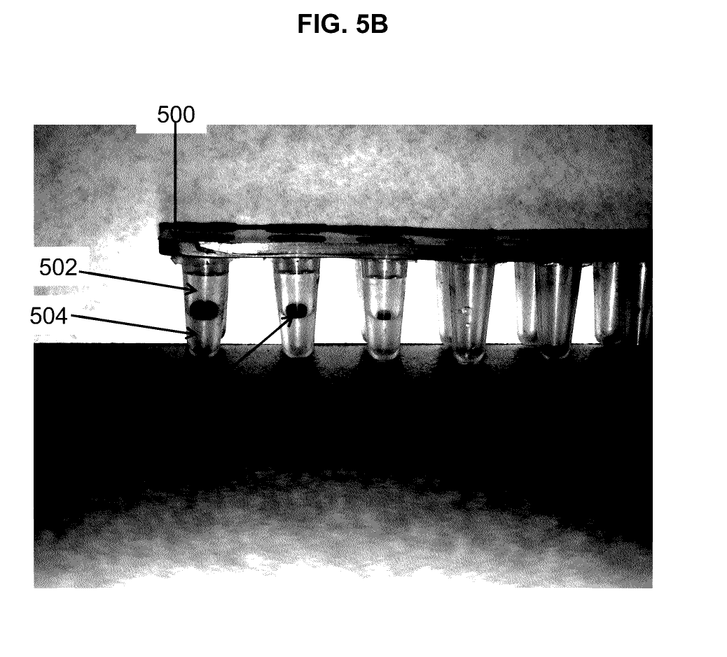

Any convenient multi-well plate formats may be utilized in the subject supports, including, but not limited to, 2-well, 4-well, 6-well, 8-well, 10-well, 12-well, 14-well, 16-well, 17-well, 96-well, 384-well or 1536 well. The pitch of the wells in the multi-well plate may vary, ranging in some instances from 3 to 20 mm, such as 4 to 15 mm, including 5 to 10 mm. In certain embodiments, the support has a multiwell format having a staggered well configuration such as or analogous to that depicted in FIG. 4, which can be expanded to any conveniently sized array of containers using the same spacing. In certain embodiments, the support is a 17-well plate that includes a 3-4-3-4-3 configuration of rows of containers and has dimensions as depicted in FIG. 4. In certain embodiments, the distance between the center of adjacent containers in a row is 6.0 mm. In certain cases, the distance from the center of the first row to the center of each following row of CLC containers is 6.0 mm. In certain embodiments, each container is cylindrical with a flat bottom where the depth of the container is 7.5 mm and the diameter is 3.5 mm. In certain embodiments, the support has a multiwell format having a parallel well row configuration such as or analogous to that depicted in FIG. 5A, which can be expanded to any conveniently sized array of containers using the same spacing. In certain embodiments, the support is a 16-well plate that includes an 8-well double row of containers configuration and has dimensions as depicted in FIGS. 5A. In certain embodiments, the distance between the center of adjacent containers in a row is 9.0 mm. In certain cases, the distance from the center of the first row to the center of each following row of CLC containers is 9.0 mm. In certain embodiments, each container is conical with a curved bottom where the depth of the container is 12 mm, e.g., as shown in FIG. 5A.

In some cases, the support includes a plurality of CLC containers configured according to SLAS (Society for Laboratory Automation and Screening) standards for a microplate. In some instances, the outside dimension of the microplate has a length of 127.76±0.5 mm and a width of 85.48±0.5 mm. In certain cases, the support is a 96-well plate. In some cases, the support is a 384-well plate.

In certain instances, the support is a 96-well plate that includes a configuration of eight rows by twelve columns of CLC containers. In certain embodiments of a 96-well plate, the distance between the left outside edge of the plate and the center of the first column of CLC containers is 14.38±0.7 mm and the center of each following column of CLC containers is an additional 9.0±0.7 mm in distance from the left outside edge of the plate; and the distance between the top outside edge of the plate and the center of the first row of CLC containers is about 11.24±0.7 mm and the center of each following row of CLC containers is an additional 9.0±0.7 mm in distance from the top outside edge of the plate. In certain embodiments, the plate height is 14.35±0.25 mm.

In certain instances, the support is a 384-well plate that includes a configuration of 16 rows by 24 columns of CLC containers. In certain embodiments of the 384-well plate, the distance between the left outside edge of the plate and the center of the first column of CLC containers is 12.13±0.7 mm and the center of each following column of CLC containers is an additional 4.5±0.7 mm in distance from the left outside edge of the plate; and the distance between the top outside edge of the plate and the center of the first row of CLC containers is about 8.99±0.7 mm and the center of each following row of CLC containers is an additional 4.5±0.7 mm in distance from the top outside edge of the plate.

A variety of well types, shapes and sizes may be utilized. In some instances, each of the plurality of CLC containers in the multiwell plate (e.g., the 96-well or 384-well plates described herein) is a discrete chimney well. In some cases, each of the plurality of CLC containers in the multiwell plate is a discrete rounded well.

In some instances, the average well diameter of the multiwell plate ranges from 2.0 to 10 mm, such as 4.5 to 8.0 mm, such as from 5.0 to 7.0 mm. In certain instances, the well volume ranges from 190 μL to 400 μL. Any convenient well volumes may be utilized in a 96-well plate according to the particular application, including but not limited to, 190 μL, 205 μL, 300 μL, 320 μL and 360 μL volumes.

In some embodiments, the average well diameter of the multiwell plate ranges from 2.0 to 4.0 mm, such as from 2.6 to 3.6 mm. In certain embodiments, the well volume ranges from 30 μL to 190 μL. Any convenient well volumes may be utilized in a 384-well plate according to the particular application, including but not limited to, 35 μL, 50 μL, 90 μL, 112 μL, 180 μL.

The subject supports may also be configured to provide for optical interrogation of the CLC contained in the plurality of containers. In some instances, the bottom of each well of the plurality of CLC containers in the multiwell plate is transparent. Such a configuration may provide for optical interrogation of the CLC from the bottom surface of the support. In some cases, each of the plurality of CLC containers in the multiwell plate is opaque. Such a configuration may provide for reduced backgrounds and light contamination between wells, e.g., in applications involving luminescent interrogation.

The subject supports may also be configured to provide for heating of individual CLC containers, e.g., as described in the subject systems. In some cases, discrete CLC containers are configured to the thermally cycled using any convenient thermoelectric devices and methods. Such supports may find use in a variety of applications and methods, e.g., applications involving DNA amplification that make use of the Polymerase Chain Reaction (PCR).

### Systems

As summarized above, aspects of the invention include systems made up of a CLC support having a CLC present in one or more CLC containers thereof, e.g., a multiplexed CLC system. The systems may include a CLC support (e.g., as described above) and a CLC disposed in or nor more of the CLC containers of the support. As summarized above, in some cases, a CLC of the subject systems includes a fluorous carrier liquid (e.g., as described herein) and an encapsulating liquid that is immiscible with the fluorous carrier liquid (e.g., as described herein) and is disposed on a free surface of the fluorous carrier liquid. The CLC further includes a core aqueous sample liquid. In some instances, the system includes a plurality of CLCs disposed in the plurality of CLC containers. This multiplexed configuration of discrete contained CLCs finds use in a variety of applications. In some instances, the supports are multi-well plates that are reusable. By reusable is meant that following the preparation and use (e.g., as described herein) of a CLC, the CLC may be removed from the container and the support reused. Since the aqueous sample liquid does not wet the inner surface of the subject CLC containers, there is low or no carryover of sample when the support is reused. In some cases, the discrete CLC containers of the support are sealable which may provide for desirable storage stability and/or minimize evaporative loss of liquids from the container, e.g., during heating.

In some instances, the aqueous sample has a density between that of the carrier liquid and the encapsulating liquid of the CLC. The carrier liquid in some cases has a density higher than that of the encapsulating liquid of the CLC. In certain instances, values of densities for the fluids involved range from 1,300 to 2,000 kg/m³ for the carrier liquid, from 700 to 990 kg/m³ for the immiscible encapsulating liquid, such as approximately 920 kg/m³, and from 900 to 1200 kg/m³ for the aqueous sample. In certain embodiments, the carrier liquid has a density in the range of from 1,800 to 2,000 kg/m³, such as approximately 1,900 kg/m³. In some embodiments, the encapsulating liquid has a density in the range of from 700 to 990 kg/m³, such as approximately 920 kg/m³. In certain cases, the aqueous liquid has a density of approximately 1000 kg/m³. An example of one such set of operating liquids and densities includes, but is not limited to: a carrier liquid that is a fluorocarbonated oil (e.g., Fluorinert FC-40) having a density of approximately 1,900 kg/m³; an encapsulating liquid of the CLC that is a silicone oil (e.g., phenylmethylpolysiloxane) having a density of approximately 920 kg/m³; and the aqueous sample liquid having a density of approximately 1000 kg/m³.

### Carrier Liquid

In some embodiments, the carrier liquid of a CLC is a fluorous carrier liquid. A variety of fluorous solvents and liquids may be utilized in the subject CLCs as a carrier liquid. In some instances, the fluorous carrier liquid is a perfluorinated amine oil. In certain embodiments, the fluorous carrier liquid is a perfluorocarbon. In certain embodiments, the fluorous carrier liquid is a fluorohydrocarbon. In certain embodiments, the fluorous carrier liquid is a hydrofluoroether (HFE). In certain embodiments, the fluorous carrier liquid is a fluorocarbonated oil (e.g., Fluorinert FC-40). In certain cases, the fluorous carrier liquid is a perfluorinated alkyl-substituted heterocycle. Fluorous liquids of interest include, but are not limited to, Fluorinert FC-40, Fluorinert FC-43, Fluorinert FC-70, Fluorinert FC-72, Fluorinert FC-75, Fluorinert FC-770, Fluorinert FC-3283, Fluorinert FC-3284, Fomblin HC PFPE, Galden PFPE, Solvera PFPE, and Krytox. In certain instances, the carrier liquid is Fluorinert FC-40. Perfluorocarbons of interest include, but are not limited to, perfluorohexane, perfluoromethylcyclohexane and perfluorodecalin. Hydrofluoroethers of interest include, but are not limited to, nonafluorobutyl methyl ether (e.g., HFE-7100). In some embodiments, the fluorous carrier liquid is methoxyperfluorobutane.

### Encapsulating Liquid

Any convenient encapsulating liquids which are immiscible with the carrier liquid may be used in the subject CLCs. The encapsulating liquid is also immiscible with the aqueous sample liquid which it encapsulates in the CLC. In some cases, the encapsulating liquid is less dense than the carrier liquid so that the encapsulating liquid may be easily disposed on the top surface of the carrier liquid in the contained CLC. In certain cases, the density of the encapsulating liquid is less than the density of the aqueous sample liquid (e.g., as described herein).

In certain embodiments, the encapsulating liquid is non-fluorous. In certain embodiments, the encapsulating liquid is a silicone oil. In certain embodiments, the encapsulating liquid is a mineral oil. In certain embodiments, the encapsulating liquid is a paraffin oil. Encapsulating liquids of interest include, but are not limited to, phenylmethylpolysiloxane, silicone surfactants, cross-linked silicone surfactants, silicone elastomers, silicone resins, silicone gums, amine-functionalized silicone, dimethicone, phenyl dimethicone, diphenyl dimethicone, phenyl trimethicone, trimethylsiloxyphenyl dimethicone, alkyl dimethicones such as cetyl dimethicone, and mixtures thereof.

Silicone surfactants of interest include, but are not limited to, those sold by Dow Corning under the tradename 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone. Polyoxyalkylenated silicone elastomers that may be used include, but are not limited to, those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer. In certain embodiments, the encapsulating liquid is a phenylmethylpolysiloxane-based oil.

Any convenient additives may be included in the subject encapsulating liquid and/or carrier liquid, including but not limited to, surfactants, pigments, antioxidants, stabilizers, etc. Such additives may provide for a desirable property such as an optical property or a change in density. Additives of interest include, but are not limited to, polysorbates, SPAN 80, SPAN 65, Tween 20 and the like. In some embodiments, the encapsulating liquid includes a phenylmethylpolysiloxane-based oil and a polysorbate additive. The additives may have a hydrophilic-lipophilic balance number in the range of 2 to 8. The hydrophilic-lipophilic balance of an additive is a measure of the degree to which it is hydrophilic or lipophilic, using the Griffin method. In some cases, the combined total hydrophilic-lipophilic balance number of the additives is in the range of 2 to 8. In some cases, the total additives within the encapsulating liquid range between 0.001% and 10% by weight.

### CLC Samples

Aspects of the subject systems include a CLC micro-reactor that is contained in the encapsulating liquid of the CLC. In some embodiments, the micro-reactor comprises an aqueous sample liquid. Any convenient samples may be included in the aqueous liquid of the micro-reactor depending on the application of interest in which the subject supports and systems find use. As used herein, the terms “micro-reactor” and “aqueous sample liquid” are used interchangeably to refer to the aqueous media contained in the CLC which may be manipulated according to a particular application of interest.

The term “sample” as used herein refers to a material or mixture of materials, in some cases in liquid form, containing one or more analytes of interest. In some cases, the analyte is a biomolecule, such as a nucleic acid, a sugar, a lipid, a protein, a peptide, etc. In one embodiment, the term as used in its broadest sense, refers to any plant, animal or bacterial material containing cells or biomolecules of interest, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. A “biological sample” can refer to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. In certain embodiments, the sample has been removed from an animal or plant. Biological samples of the invention include cells. The term “cells” is used in its conventional sense to refer to the basic structural unit of living organisms, both eukaryotic and prokaryotic, having at least a nucleus and a cell membrane. In certain embodiments, cells include prokaryotic cells, such as from bacteria. In other embodiments, cells include eukaryotic cells, such as cells obtained from biological samples from animals, plants or fungi. In some embodiments, the micro-reactor includes a particle suspension in aqueous media.

### CLC Manipulation Devices

As summarized above, aspects of the invention include CLC manipulation devices for CLCs. The subject devices may include all components necessary for preparing, containing, and manipulating CLCs (e.g., as described herein) contained in the subject CLC support (e.g., as described here). Additional details regarding CLC manipulation devices that may be configured to manipulate CLC supports include those described in in U.S. Pat. Nos. 8,465,707 and 9,080,208; as well as United States Patent Application Publication No. 20140371107; and Published PCT Application Nos: WO2014/083435; WO2014/188281; WO2014/207577; WO2015/075563; WO2015/075560; the disclosures of which applications are herein incorporated by reference.

In some cases, the device is an automated multiwell plate handling device that include at least on plate locations at which a multwell CLC support may be disposed. The devices include all liquid handling and other components necessary to prepare an array of composite liquid cells (CLCs), as reviewed in greater detail below. The devices may include a robotically controlled liquid handler for delivering liquids, samples and/or reagents of interest to each container of the multiwell support. The devices are automated, in that they are configured so that at least some, if not all, steps of a given protocol may occur without human intervention, beyond introduction of the liquid components into the device, loading of any requisite reagents and input of information, and activating the device to perform the steps of the method. Steps of a protocol that may be automated in the devices include, but are not limited to: liquid transfer steps, reagent addition steps, thermal cycling steps, product purification steps, etc.

In some embodiments, the device includes all components necessary to prepare a nucleic acid library suitable for next generation sequencing (NGS) from an initial nucleic acid sample. Accordingly, the devices are configured such that an initial nucleic acid sample can be introduced into the device and a complete nucleic acid library ready for use in a next generation sequencing protocol can be obtained from the device, with little if any user interaction with the device between the time of sample introduction and product NGS library retrieval.

Devices according to embodiments of the invention include at least a thermal chip module, one or more plate locations, a robotically controlled liquid handler configured to transfer liquid between the one or more plate locations, the at least one thermal chip module and a bulk reagent dispenser configured to access each node of the at least one thermal chip module. Each of these components or subunits of the device will now be described in greater detail.

### Thermal Module

As summarized above, devices described herein include a thermal module. The devices may include a single thermal module, or two thermal modules. Thermal modules are plate or chip type structures that include one or more nodes, where each node is configured to have thermal contact with a CLC container of the multiwell plate positioned at the node. In some embodiments, the number of nodes is 96 or 384, e.g., in embodiments where correspondence with conventional multi-well plates is desired. Thermal modules may be made of thermally conductive material. Materials of interest include, but are not limited to thermally conductive materials, e.g., composites, ceramics, and metals, including aluminum. The thermal module may be configured to accommodate a CLC support plate.

An aspect of the thermal modules is that they are thermally controlled, such that the temperature of the environment (and therefore experienced by a CLC in a CLC support accommodated by the thermal module) may be controlled, e.g., including precisely controlled, e.g., to a tenth of degree or better. The range of temperature control may vary, where in some instances the temperature may be controlled between 4 to 120° C., such as 4 to 98° C. To provide for thermal control, the thermal module may include heating and/or cooling elements. For example, the thermal module may include a cooling region configured to be operably attached to temperature modulator, e.g., a thermoelectric module, a fluidic cooling system or a forced convection cooling system. The module may also include a heating element in thermal contact with the CLC support. Heating stabilization features are further described in WO/2014/188281.

Aspects of the present disclosure include CLC supports having discrete CLC containers, by comparison to CLC technology which includes a common carrier oil to carry multiple CLCs. The discrete CLC containers of the subject supports find use in conjunction with thermal cyclers which provide for individual thermal control of the discrete CLC containers. The CLCs of the subject systems may be thermally cycled (e.g. in applications involving PCR) using any convenient thermoelectric driven approach.

### Detector Module

The device may include a detector module configured to allow for CLC optical interrogation. In some instance, a line of sight from a detector to a CLC is maintained through the CLC support. In certain cases, The CLC support is opaque and a line of sight is maintained from the top via an open container. When the CLC support is transparent, optical interrogation may be performed from the bottom or the top of the well. Optical detection methods include, but are not limited to, fluorescence, absorbance, Raman, interferometry and shadowgraphy.

Devices described herein include one or more plate locations. While the number of plate locations present in the device may vary, in some instances the device includes 1 to 10 plate locations, such as 2 to 8 plate locations, e.g., 6 plate locations. The plate location(s) may be arranged in any convenient manner in the device, where in some instances in which the device includes a plurality of plate locations, the plurality of plate locations are arranged adjacent to each other. Plate locations are regions or areas of the device configured to hold a laboratory plate, such as a multi-well plate, e.g., a 96 or 384 multi-well plate, or analogous structure, e.g., a test tube holder or rack, etc. A given plate location may be a simple stage or support configured to hold a laboratory plate. While the dimensions of the plate locations may vary, in some instances the plate locations will have a planar surface configured to stably associate with a laboratory plate, where the planar surface may have an area ranging from 10 mm to 400 mm, such as 10 mm to 200 mm. The planar surface may have any convenient shape, e.g., circular, rectangular (including square), triangular, oval, etc., as desired. To provide for stable association between a plate location and a research plate, the plate location may include one or more stable association elements, e.g., clips, alignment posts, etc.

In some instances, a given plate location may be configured to be agitated, i.e., the plate location is a shaker unit. As such, it may include an agitator (e.g., vibrator or shaker component). While the frequency of the movement of the plate location provided by the agitator component may vary, in some instances that agitator may be configured to move the plate location between first and second positions at a frequency ranging from 1 rpm to 4000 rpm, such as 50 rpm to 2500 rpm, where the distance between the first and second positions may vary, and in some instances ranges from 10 mm to 400 mm, such as 25 mm to 100 mm.

### Robotically Controlled Liquid Handler

Devices described herein may include a robotically controlled liquid handler. The robotically controlled liquid handler is a unit that is configured to transfer liquid and/or CLCs between various locations of the device, such as the plate location(s). In some instances, the robotically controlled liquid handler comprises interchangeable heads configured for sample dispensing, vacuum and purification tasks. In a general sense, the robotic liquid handler may be any liquid handling unit that is capable of transferring a quantity of liquid between two distinct locations of the device, such as between plate locations. Robotic liquid handlers of interest are ones that can remove a defined volume of liquid from a first location of the device, such as a well of a laboratory plate, and deposit that volume of liquid at second location of the device, e.g., a product collection location. While the volume of liquid that the handler is configured to transfer may vary, in some instances the volume ranges from 100 nl to 10 ml, such as 100 nl to 1 ml. Further details regarding capillary liquid handling systems that may be employed in the subject device are provided in WO 2014/08345; the disclosure of which is herein incorporated by reference.

Of interest are robotic liquid handling systems that are further configured for making and processing CLCs, e.g., in CLC mediated NGS library production protocols. In such embodiments, the liquid handling system may include a CLC forming component like the one described in detail in U.S. Pat. No. 8,465,707, the disclosure of which is herein incorporated by reference.

### Bulk Reagent Dispenser

Devices described herein may include a bulk reagent dispenser. The bulk reagent dispenser is an automated reagent dispenser that is configured to deposit a metered volume of a reagent composition, e.g., a liquid reagent composition, into the containers of a CLC support plate. In some instances, the bulk reagent dispenser is configured to deposit a metered volume of a reagent composition, e.g., polymerase, nucleotide mix, primer, adapter, buffer, ligase etc. In some instances, the bulk reagent dispenser includes a reagent metering element (such as a liquid reagent metering unit) operatively coupled to a bulk reagent source (such as a liquid reagent reservoir, e.g., present in a cartridge) by an automated movement arm, e.g., an arm that is configured to move in the X and/or Y and/or Z directions. In some instances, the bulk reagent dispenser is configured to be able to individually introduce a metered amount of a reagent composition into a container and any CLC present therein in a non-contact microfluidic dispensing manner, e.g., by dropping an amount of the reagent composition onto a CLC such that the reagent composition merges with the CLC in the node.

Devices described herein may include a fluidics module that includes one or more liquid reservoirs, e.g., for system fluids, waste collection, etc. System fluids of interest include, but are not limited to, wash fluids, elution fluids, etc. Where desired, the waste collection reservoir is operatively coupled to a single waste drain.

Devices described herein may be configured to automatically produce large numbers of libraries in a short period of time following commencement of a given library preparation run. The numbers of library samples that the devices may be configured to simultaneously produce ranges in some instances from 1 to 1000, such as 8 to 768, e.g., 96, 192, 384 or 768 libraries. While the amount of time required to produce such libraries may vary, in some instances the amount of time ranges from 1 hour to 48 hours, such as 2 to 36 hours, e.g., 6 hours. To facilitate reagent handling and device set up, the device may include a control processor in operative communication with a handheld unique identifier (e.g., barcode) scanner, which scanner may communicate with the processor via a wired or wireless communication protocol. Such embodiments may be used to upload identifying information regarding laboratory plates and/or reagent sources into the control processor of the device in order configure the device to automatically perform a library preparation protocol.

### Methods

Aspects of the present disclosure include methods of preparing an array of composite liquid cells (CLCs) in the subject support (e.g., as described herein). Aspects of the methods include introducing both a carrier liquid and an encapsulating liquid to a container of a support to produce a CLC in the container. The carrier liquid and encapsulating liquid may be introduced simultaneously or sequentially in any convenient order. The densities of the liquids may be selected to provide for a desirable configuration of the two immiscible liquids in the subject CLC container (e.g., as described herein). In some instances, the method includes introducing a carrier liquid to an empty container to provide for a volume of carrier liquid in contact with the inner surface of the container and having a concave meniscus. In some cases, the method further includes, introducing an encapsulating liquid to the top surface of the carrier liquid in the container to provide an encapsulating liquid droplet having a volume suitable for containing the aqueous sample liquid of interest. The volumes of carrier and encapsulating liquids introduced to the CLC containers may be selected according to a variety of factors, such as the volume of the container, the diameter of the container, the volume of sample liquid, and the application of interest. In certain cases, each CLC container of the system receives the same volumes of liquids to provide for a consistently located micro-reactor in each CLC.

The method may further include introducing a sample liquid into the CLC. These liquid handling steps may be achieved in a variety of ways, including but not limited to, via operation of an automated liquid handling system, e.g., as described herein, or via use of the CLC liquid handling methods described in U.S. Pat. Nos. 8,465,707 and 9,080,208; as well as United States Patent Application Publication No. 20140371107; and Published PCT Application Nos: WO2014/083435; WO2014/188281; WO2014/207577; WO2015/075563; WO2015/075560; the disclosures of which applications are herein incorporated by reference.

The methods and systems described herein find use in a variety of different applications. Applications in which the methods and systems find use include CLC mediated protocols, including but not limited to those described in U.S. Pat. Nos. 8,465,707 and 9,080,208; as well as United States Patent Application Publication No. 20140371107; and Published PCT Application Nos: WO2014/083435; WO2014/188281; WO2014/207577; WO2015/075563; WO2015/075560; the disclosures of which applications are herein incorporated by reference.

Aspects of the present invention include methods of producing a nucleic acid library, e.g. an array of nucleic acids, or mixtures thereof, disposed in the micro-reactors of multiple CLCs contained in the subject supports. A variety of nucleic acid libraries may be prepared according to the subject methods making use of the subject CLC systems. In certain embodiments, the method is a method of producing a next generation sequencing (NGS) library from an initial nucleic acid sample by using a device of the present disclosure, e.g., as described above, in a CLC mediated library preparation protocol. The devices of the invention may be employed to produce NGS libraries suitable for sequencing in a variety of different NGS platforms, including but not limited to: the HiSeg™ MiSeg™ and Genome Analyzer™ sequencing systems from Illumina®; the Ion PGM™ and Ion Proton™ sequencing systems from Ion Torrent™; the PACBIO RS II sequencing system from Pacific Biosciences, the SOLiD sequencing systems from Life Technologies™, the 454 GS FLX+ and GS Junior sequencing systems from Roche, or any other sequencing platform of interest.

In preparing an NGS library, a nucleic acid sample from which the library is to be prepared is first provided. Any convenient nucleic acid sample preparation method may be employed. Nucleic acid sample preparation may include fragmenting an initial nucleic acid source sample to produce a fragmented nucleic acid sample made up of nucleic acid fragments of suitable size for sequencing with a given NGS sequencing platform. Source nucleic acids of interest include, but are not limited to: deoxyribonucleic acids, e.g., genomic DNA, complementary DNA (or “cDNA”, synthesized from any RNA or DNA of interest), recombinant DNA (e.g., plasmid DNA); ribonucleic acids, e.g., messenger RNA (mRNA), a microRNA (miRNA), a small interfering RNA (sRNA), a transacting small interfering RNA (ta-sRNA), a natural small interfering RNA (nat-sRNA), a ribosomal RNA (rRNA), a transfer RNA (tRNA), a small nucleolar RNA (snoRNA), a small nuclear RNA (snRNA), a long non-coding RNA (IncRNA), a non-coding RNA (ncRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared sRNA (esiRNA), a small temporal RNA (stRNA), a signal recognition RNA, a telomere RNA, a ribozyme; etc.

Source nucleic acids may be fragmented using any convenient protocol, e.g., passing the sample one or more times through a micropipette tip or fine-gauge needle, nebulizing the sample, sonicating the sample (e.g., using a focused-ultrasonicator by Covaris, Inc. (Woburn, Mass.)), bead-mediated shearing, enzymatic shearing (e.g., using one or more RNA-shearing enzymes), chemical based fragmentation, e.g., using divalent cations, fragmentation buffer (which may be used in combination with heat) or any other suitable approach for shearing/fragmenting an initial nucleic acid to generate a shorter template nucleic acids suitable for NGS library preparation. In certain aspects, the template nucleic acids generated by shearing/fragmentation of a starting nucleic acid sample has a length of from 10 to 20 nucleotides, from 20 to 30 nucleotides, from 30 to 40 nucleotides, from 40 to 50 nucleotides, from 50 to 60 nucleotides, from 60 to 70 nucleotides, from 70 to 80 nucleotides, from 80 to 90 nucleotides, from 90 to 100 nucleotides, from 100 to 150 nucleotides, from 150 to 200, from 200 to 250 nucleotides in length, or from 200 to 1000 nucleotides or even from 1000 to 10,000 nucleotides, for example, as appropriate for the sequencing platform chosen.

The CLCs of the subject system may be loaded with nucleic acid sample(s). Common reagents may be dispensed as needed during the library preparation procedure into each CLC container, including, but not limited to: dNTPs (e.g., in the form of a mastermix), enzymes, e.g., polymerases, primers, platform specific sequencing adaptors (which may or may not be integrated with the primers), nucleic acid barcodes, ligases, etc. In some cases, a bulk reagent dispenser may be employed in a non-contact microfluidic dispensing protocol in order to add the reagents to the CLCs. Each reagent may be sequentially added, or two or more reagents may be pre-combined and added to the CLCs, as desired. Following or during reagent addition to the CLCs, the thermal chip module may be subjected to temperature modulation, e.g., in the form of thermal cycling of the discrete containers of the CLC system, as desired for a given NGS library preparation protocol. A variety of washing and purification protocols may be adapted for use in the subject methods. Details regarding CLC production methods which may be employed are further described in U.S. Pat. No. 8,465,707, the disclosure of which is herein incorporated by reference. Details regarding magnetic bead/conduit based purification protocols that may be employed are further described in PCT Application Serial No. PCT/IB2014/002159 published as WO 2014/207577; the disclosure of which is herein incorporated by reference.

The resultant product NGS libraries may then be sequenced, as desired, using any convenient NGS sequencing platform, including: the HiSeq™, MiSeq™ and Genome Analyzer™ sequencing systems from Illumina®; the Ion PGM™ and Ion Proton™ sequencing systems from Ion Torrent™; the PACBIO RS II sequencing system from Pacific Biosciences, the SOLiD sequencing systems from Life Technologies™, the 454 GS FLX+ and GS Junior sequencing systems from Roche, or any other convenient sequencing platform.

Aspects of the present disclosure include methods of assaying a sample of a CLC present in a container of a support (e.g., as described herein). The sample may include or be suspected of including an analyte of interest. The sample may include any convenient analytes to be assayed. Analytes of interest include, but are not limited to, nucleic acids, proteins (e.g., enzymes, target proteins, antibodies), peptides, lipids, carbohydrates, hormones, drugs, ligands, etc.

Assay protocols of interest which may be adapted to be performed in the subject CLC systems according to the subject methods include any convenient methods where small samples volumes, recovery of precious samples, minimization of use of reagents, minimization of carryover or contamination of supports are of interest. In some instances, the subject methods are performed using sample volumes of 10 μL or less, such as 5 μL or less, 2 μL or less 1 μL or less, 900 nL or less, 800 nL or less, 700 nL or less, 600 nL or less, 500 nL or less, 400 nL or less, 300 nL or less, 200 nL or less, 100 nL or less, or even less. Any convenient reagents may be introduced into the CLC according to the steps if an assay protocol of interest. Reagents of interest include, but are not limited to, enzyme substrates, primers, dNTPs, enzymes (e.g., polymerases, ligases, HRP, alkaline phosphatase etc.), nucleic acids, antibody conjugates, chemoselective imaging agents, and the like. In some instances of the method, the CLC includes a sample liquid comprising a nucleic acid ligation reaction mixture. In some instances of the method, the CLC includes a sample liquid comprising a polymerase chain reaction sample, and the method further includes thermally cycling the sample to amplify a nucleic acid.

### Kits

Aspects of the present disclosure also include kits. The kits may include, e.g., one or more CLC supports, e.g., as described above. Where desired, the kits may further include one or more additional components that find use in a CLC application, e.g., reagents, buffers, etc. Any or all of the kit components may be present in sterile packaging, as desired.

In addition to the above-mentioned components, a subject kit may further include instructions for using the components of the kit, e.g., to practice the subject methods. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., a portable flash drive, CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

### Preparation of an Injection Molded Fluoropolymer Support

A 17-well support was designed including a staggered arrangement of three 3-well columns and two 4-well columns of CLC containers. FIG. 4 shows a schematic detailing the dimensions of the exemplary 17-well multiwell support. Unless otherwise specified, dimensions and tolerances are in millimeters. Tolerances are as follows: ±0.1 mm, ≦±0.5°.

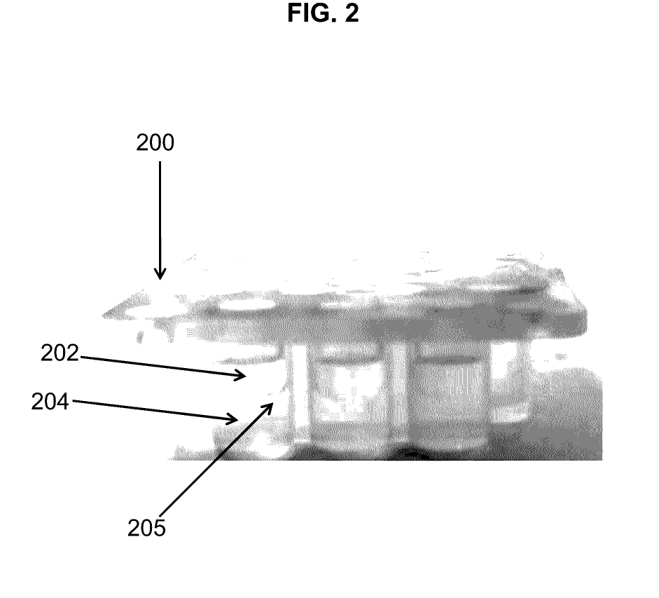

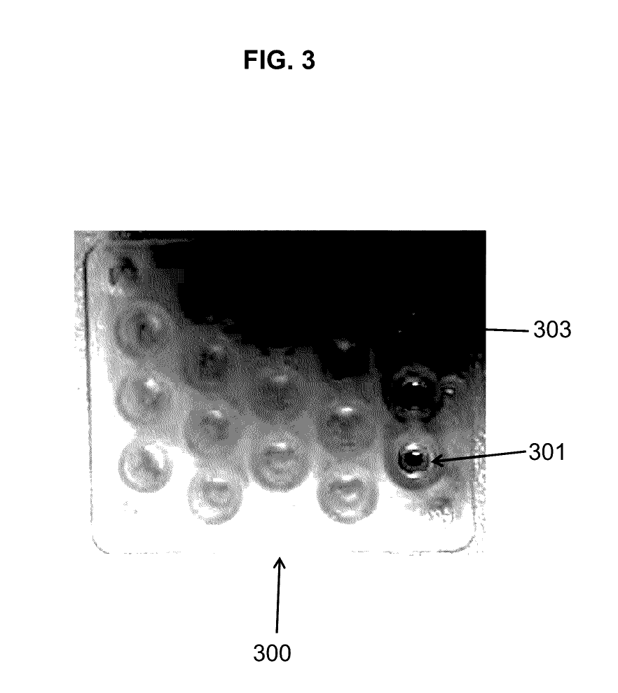

A transparent sample plate was prepared via an injection molding procedure using Daikiin NEOFLON™ PFA AP-201 SH fluoropolymer. See FIGS. 2 and 3 which show images of the fluoropolymer multiwell plate produced. An opaque version of the exemplary multiwell plate was also prepared using the same method except a black colorant was added to the fluoropolymer during fabrication.

### Example 2

### Preparation of a CLC

Carrier liquid and encapsulating liquid were added to various wells of the support. FIG. 2 shows an image of an exemplary multiwell CLC support (200) includes an encapsulating liquid (202) disposed on the top surface of a carrier liquid (204) whose interface defines a visible concave meniscus (205). The fluorous carrier fluid used is FC-43 (3M). The encapsulating silicone oil used was PD5 (Momentive). FIG. 2 shows a CLC comprising of a volume of FC-43 and a volume of PD5 held in an injection moulded CLC support fabricated in PFA (Daiken AP-201 SH).

A sample of an aqueous solution including a colored dye to aid in visualization was added to various wells. FIG. 3 shows an image of the multiwell CLC system (300) with an aqueous sample liquid in place (303) in the encapsulating liquid of the CLC in three wells. The image shows that the aqueous sample liquid was self-centered in the container away from the inner surface (301) of the CLC container.

### Example 3

### Preparation of an Injection Molded Fluoropolymer Support

An 16-well support was designed including a two-row arrangement of 8 CLC containers was prepared. FIG. 5A shows a schematic detailing the dimensions of the exemplary 16-well multiwell support. Unless otherwise specified, dimensions and tolerances are in millimeters. Tolerances are as follows: ±0.1 mm, ≦±0.5°.

Carrier liquid and encapsulating liquid were added to various wells of the support. FIG. 5B shows an image of an exemplary multiwell CLC support (500) includes an encapsulating liquid (502) disposed on the top surface of a carrier liquid (504) whose interface defines a visible concave meniscus. A sample of an aqueous solution including a colored dye to aid in visualization was added to various wells. FIG. 5B shows an image of the multiwell CLC system (500) with an aqueous sample liquid in place (505) in the encapsulating liquid of the CLC in three wells. The image shows that the aqueous sample liquid was self-centered in the container away from the inner surface of the CLC container.

### Example 4

### Contact Angle Measurements

FIG. 6 shows the results of contact angle measurements of a material of interest that finds use in exemplary CLC supports. FEP, a common fluoropolymer was used as a reference material as it was known to provide the appropriate surface properties for CLCs. Contact angle measurements were taken using the Sessile drop technique. Measurements were taken using a Dataphysics Instruments OCA 20 system. The results from this study were used to define the contact angles between a fluorous carrier fluid (GC1) and the housing material and between the encapsulating silicone oil (GC2) and the housing material. The moulded (injection moulded polypropylene) data set was known to yield the incorrect meniscus shape. Analysis of the two data sets enabled inference of the critical surface properties required for a CLC support.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A composite liquid cell (CLC) support, the support comprising:
a plurality of CLC containers, wherein each CLC container is configured to hold a CLC and comprises a fluorophilic inner surface having a water contact angle of 80 degrees or more.

2. The support according to claim 1, wherein the fluorophilic inner surface has a first contact angle with a fluorous carrier liquid; and a second contact angle with an encapsulating liquid that is immiscible with the carrier liquid, wherein the first contact angle is less than the second contact angle and the fluorous carrier liquid is more dense than the encapsulating liquid.

3-4. (canceled)

5. The support according to claim 1, wherein the fluorophilic inner surface comprises a fluoropolymer composition.

6. The support according to claim 5, wherein the fluoropolymer composition comprises a melt processible cofluoropolymer.

7. (canceled)

8. The support according to claim 5, wherein the fluoropolymer composition further comprises a pigment or a black colorant to provide for an opaque support.

9. The support according to claim 1, wherein the support has an integrated structure made up of a homogeneous material.

10. The support according to claim 1, wherein the support comprises a multiwell format.

11. (canceled)

12. The support according to claim 1, wherein the immiscible encapsulating liquid is a silicone oil.

13. The support according to claim 1, wherein the plurality of CLC containers is configured according to SLAS (Society for Laboratory Automation and Screening) standards for a microplate.

14-23. (canceled)

24. The support according to claim 1, wherein the bottom of each of the plurality of CLC containers is transparent.

25. The support according to claim 1, wherein each of the plurality of CLC containers is opaque.

26. A system for multiplexed composite liquid cells (CLCs), the system comprising:
a CLC support comprising a plurality of CLC containers, wherein each CLC container is configured to hold a CLC and comprises a fluorophilic inner surface; and
a CLC disposed in at least one CLC container and comprising:
a fluorous carrier liquid; and
an encapsulating liquid that is immiscible with the fluorous carrier liquid and is disposed on a free surface of the fluorous carrier liquid;
wherein the fluorophilic inner surface has a water contact angle of 80 degrees or more.

27-49. (canceled)

50. A method of preparing an array of composite liquid cells (CLCs), the method comprising:
introducing a carrier liquid to a container of a support of claim 1; and
introducing an encapsulating liquid to the container to produce a CLC in the container.

51-53. (canceled)

54. A method, comprising assaying a sample of a CLC present in a container of a support according to claim 1.

55. The method according to claim 54, wherein the sample is a biological sample.

56. The method according to claim 55, wherein the sample comprises a nucleic acid, a protein, a peptide, a lipid or a carbohydrate.

57. (canceled)

58. The method according to claim 54, further comprising introducing a reagent to the CLC.

59. The method according to claim 58, wherein the reagent comprises an enzyme or a primer.

60-61. (canceled)

62. The method according to claim 54, wherein the sample comprises a nucleic acid library.

63. The method according to claim 62, wherein the nucleic acid library is a next generation sequencing (NGS) library.
